(19) United States
(12) Patent Application Publication
(10) Pub. No.: US 2024/0426806 A1
(43) Pub. Date: Dec. 26, 2024

(54) SENSOR AND SENSOR MATERIAL FOR DETECTING PATHOGEN-DERIVED VOLATILE ORGANIC COMPOUNDS

(71) Applicant: TAO TREASURES LLC DBA NANOBIOFAB, Frederick, MD (US)

(72) Inventors: Xiaonao LIU, Frederick, MD (US); Taejun KO, Clarksburg, MD (US); Buyu YEH, Frederick, MD (US)

(21) Appl. No.: 18/829,748

(22) Filed: Sep. 10, 2024

Related U.S. Application Data
(63) Continuation-in-part of application No. PCT/US2023/064185, filed on Mar. 10, 2023.
(60) Provisional application No. 63/269,151, filed on Mar. 10, 2022.

(51) Int. Cl. G01N 33/497; G01N 27/12; G01N 33/00

(52) U.S. Cl. CPC G01N 33/4977; G01N 27/127; G01N 33/0047

(57) ABSTRACT

Sensors containing one or more metal oxides as the sensor material are used for detecting pathogen-derived VOCs, e.g., VOCs emitting from infectious wounds. The metal oxides may contain one or more metal elements selected from aluminum, antimony, bismuth, boron, cerium, chromium, cobalt, copper, gold, indium, iridium, iron, lanthanum, lead, manganese, molybdenum, nickel, niobium, osmium, palladium, platinum, rhodium, ruthenium, selenium, silicon, silver, tantalum, tellurium, terbium, tin, titanium, tungsten, vanadium, ytterbium, yttrium, zinc, and zirconium. A sensor device may have one or more sensors, which detects and records a variety of VOCs that are indicative of existence of pathogens, and the pathogens are associated with dieses and/or infections.

### TECHNICAL FIELD

This application generally relates to sensor materials, sensors, and devices for detecting certain volatile organic compounds (VOCs), and more particularly to a sensor material and sensor device for detecting VOCs emitted from an infectious wound for the purpose of wound infection detection.

### BACKGROUND

Chronic cutaneous wound infections and surgical site infections present a heavy burden on the healthcare system and can lead to increased morbidity and mortality. Common pathogens associated with chronic as well as superficial and deep surgical site infections include, but are not limited to, Staphylococcus epidermidis (SE), Streptococcus pyogenes (SP), Enterococcus faecium (EF), Staphylococcus aureus (SA), Klebsiella pneumoniae (KP), Acinetobacter baumannii (AB), Pseudomonas aeruginosa (PA), Enterobacter species (ES), Escherichia coli (EC), Proteus mirabilis (PM), Serratia marcescens (SM), Enterobacter cloacae (E.cl), and Acinetobacter anitratus (AA). Current diagnostic methods of identifying and confirming infection involve culture-based methods and molecular methods. These techniques are time and resource consuming and require sample transport. Many such techniques also have limited sensitivity and specificity inherent to sample processing and user error (requiring complex laboratory science experience and equipment). Thus, the technological limitations of these approaches can delay diagnostics, often resulting in empirical treatment before confirmation of the infectious agent, increasing the risk for sub-optimal choice of antibiotics and contributing to the development of antibiotic resistance. To improve wound infection management either in the field or in the hospital setting, it is highly desirable to have a technology that detects the development of infection directly on the wound bed and a simultaneous identification of the active microorganism.

Volatile organic compounds (VOCs) include a diverse group of carbon-based molecules, including alcohols, isocyanates, ketones, aldehydes, hydrocarbons and sulfides, which are volatile at ambient temperatures. VOC detection has the advantage of being painless, non-invasive and reproducible. There is increasing evidence that VOCs and combinations thereof are unique to various disease states and their early detection could represent a useful means of diagnosis. VOCs have been identified as potential biomarkers in diagnosis of lung cancer, breast cancer, asthma, and diabetes. Pathogens also produces VOCs. The ability to rapidly detect microbial VOCs allows identification of certain pathogens.

There is a need for sensor materials and sensors that can detect early stages of infection before symptoms develop and enable consistent monitoring through all phases of infection.

### SUMMARY

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed disclosure. It is to be understood that the value to which the modifier “about” or “approximately” refers is itself also specifically, and preferably, disclosed.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The terms “infection” and “bacterial infection” indicates the presence and/or colonization of pathogenic bacteria in or on a subject in a number or an amount sufficient to be pathogenic, that is sufficient to cause disease, damage or harm to a subject infected with said bacterium. A subject having an infection is said to be “infected” with a pathogen. Pathogenic bacteria or short “pathogens” as used herein are bacteria that are known to cause bacterial infections in subjects.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an exemplary embodiment,” means that a particular feature, structure or characteristic described in connection with the embodiment is included in at least one embodiment of the present disclosure. Thus, appearances of the phrases “in one embodiment,” “in an embodiment,” or “an exemplary embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the disclosure. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

According to one aspect of the current disclosure, a sensor has one or more sensor materials, each sensor material containing one or more metals selected from tin (Sn), terbium (Tb), cobalt (Co), zinc (Zn), indium (In), copper (Cu), nickel (Ni), chromium (Cr), manganese (Mn), tungsten (W), titanium (Ti), vanadium (V), iron (Fe), aluminum (Al), gallium (Ga), silver (Ag), gold (Au), palladium (Pd), rhodium (Rh), ruthenium (Ru), molybdenum (Mo), niobium (Nb), zirconium (Zr), yttrium (Y), lanthanum (La), platinum (Pt), silicon (Si), cerium (Ce), and tellurium (Te), wherein the sensor is capable of detecting one or more volatile organic compounds derived from one or more pathogens.

In some embodiments, each sensor is of a porous structure selected from mesoporous, macroporous, microporous, nanoporous, and hierarchical porous. A pore size of the sensor ranges from 0.4 to 2 nm, 2 to 50 nm, 50 nm to 200 nm, 200 nm to 500 nm, 500 nm to 1 μm, and 1 to 50 μm.

In other embodiments, each sensor material comprises a nanomaterial selected from nanosphere, nanorods, nanoporous, nanomesh, nanowire, nanodot, nanostar, nanosheet, nanoplate, nanoflake, nanotube, nano-hollow sphere, nanocube, nanocage, nanopolyhedron, nanobelt, nanocylinder, nanohelix, nanoprism, nanoribbon, nanoring, nanotetrapod, nanodumbbell, nanocrescent, nanosculpture, nanobrush, nanofiber, nanocrystal, nanowhisker, nanoscroll, and nanocapsule, etc.

In still other embodiments, each sensor material contains one or more oxides selected from CoOx, SnOx, ZnOx, NiOx, VOx, FeOx, CuOx, CrOx, InOx, TbOx, MnOx, WOx, TiOx, AlOx, GaOx, AgOx, AuOx, PdOx, RhOx, RuOx, MoOx, NbOx, ZrOx, YOx, LaOx, PtOx, SiOx, CeOx, TeOx, AgAlOx, AgAuOx, AgCoOx, AgCrOx, AgCuOx, AgFeOx, AgGaOx, AgInOx, AgMnOx, AgMoOx, AgNiOx, AgPdOx, AgPtOx, AgRhOx, AgRuOx, AgTiOx, AgVOx, AgWOx, AgZnOx, AgZrOx, AlCoOx, AlCrOx, AlCuOx, AlFeOx, AlGaOx, AlInOx, AlMnOx, AlMoOx, AlNiOx, AlPdOx, AlPtOx, AlRhOx, AlRuOx, AlTiOx, AlVOx, AlWOx, AlZnOx, AlZrOx, AuCoOx, AuCrOx, AuCuOx, AuFeOx, AuGaOx, AuInOx, AuMnOx, AuMoOx, AuNiOx, AuPdOx, AuPtOx, AuRhOx, AuRuOx, AuTiOx, AuVOx, AuWOx, AuZnOx, AuZrOx, CoCrOx, CoCuOx, CoFeOx, CoGaOx, CoInOx, CoMnOx, CoMoOx, CoNiOx, CoPdOx, CoPtOx, CoRhOx, CoRuOx, CoTiOx, CoVOx, CoWOx, CoZnOx, CoZrOx, CrCuOx, CrFeOx, CrGaOx, CrInOx, CrMnOx, CrMoOx, CrNiOx, CrPdOx, CrPtOx, CrRhOx, CrRuOx, CrTiOx, CrVOx, CrWOx, CrZnOx, CrZrOx, CuFeOx, CuGaOx, CuInOx, CuMnOx, CuMoOx, CuNiOx, CuPdOx, CuPtOx, CuRhOx, CuRuOx, CuTiOx, CuVOx, CuWOx, CuZnOx, CuZrOx, FeGaOx, FeInOx, FeMnOx, FeMoOx, FeNiOx, FePdOx, FePtOx, FeRhOx, FeRuOx, FeTiOx, FeVOx, FeWOx, FeZnOx, FeZrOx, GaInOx, GaMnOx, GaMoOx, GaNiOx, GaPdOx, GaPtOx, GaRhOx, GaRuOx, GaTiOx, GaVOx, GaWOx, GaZnOx, GaZrOx, InMnOx, InMoOx, InNiOx, InPdOx, InPtOx, InRhOx, InRuOx, AlFeTiOx, AlGaFeOx, AlGaMnOx, AlGaTiOx, AlInZnOx, AlNiZnOx, AlTiVxOx, AlVZnOx, AlZnCuOx, AlZnFeOx, AlZnMnOx, AlZnNiOx, AlZnTiOx, CoCrFeOx, CoCrMnOx, CoCrNiOx, CoCrTiOx, CoFeTiOx, CoMnNiOx, CoMnTiOx, CoNiTiOx, CoTiVxOx, CoTiZrOx, CoVZnOx, CrFeTiOx, CrMnTiOx, CrNiTiOx, FeMnTiOx, FeTiVxOx, FeTiZrOx, FeVZnOx, GaInZnOx, GaMnTiOx, GaTiZrOx, GaVZnOx, InMnZnOx, InNiZnOx, InTiZrOx, InVZnOx, MnNiZnOx, MnTiVxOx, MnTiZrOx, MnVZnOx, NiTiVxOx, NiTiZrOx, NiVZnOx, TiVZrOx, TiZrZnOx, TiZrCuOx, TiZrNiOx, TiZrCoOx, TiZrMnOx, TiZrFeOx, TiZrAlOx, TiZnCuOx, TiZnFeOx, TiZnMnOx, TiZnNiOx, TiZnCoOx, TiZnCrOx, TiZnAlOx, VZrZnOx, VZrCuOx, VZrCrOx, VZrMnOx, VZrNiOx, VZrCoOx, VZrFeOx, VZrAlOx, YAlOx, YCrOx, YFeOx, YInOx, YNiOx, YTiOx, ZnCrTiOx, ZnFeTiOx, ZnMnTiOx, ZnNiTiOx, ZnTiVxOx, ZnTiZrOx, ZrCrFeOx, ZrCrMnOx, ZrCrNiOx, ZrCrTiOx, ZrFeTiOx, ZrMnNiOx, ZrMnTiOx, ZrNiTiOx, ZrTiVxOx, ZrTiZnOx, ZrVZnOx, ZrZnCuOx, ZrZnFeOx, ZrZnMnOx, ZrZnNiOx, ZrZnCoOx, ZrZnAlOx, AlCoCrTiOx, AlCoMnTiOx, AlCoNbTiOx, AlCoTaTiOx, AlCoVTiOx, AlFeCoTiOx, AlFeCrTiOx, AlFeMnTiOx, AlFeMoTiOx, AlFeNbTiOx, AlFeTaTiOx, AlFeVTiOx, AlFeZnTiOx, AlNiCoTiOx, AlNiCrTiOx, AlNiMnTiOx, AlNiMoTiOx, AlNiNbTiOx, AlNiTaTiOx, AlNiVTiOx, AlNiZnTiOx, CuCoAlTiOx, CuCoCrTiOx, CuCoMnTiOx, CuCoMoTiOx, CuCoNbTiOx, CuCoTaTiOx, CuCoVTiOx, CuCoZnTiOx, CuFeAlTiOx, CuFeCrTiOx, CuFeMnTiOx, CuFeMoTiOx, CuFeNbTiOx, CuFeTaTiOx, CuFeVTiOx, CuFeZnTiOx, CuNiAlTiOx, CuNiCoTiOx, CuNiCrTiOx, CuNiMnTiOx, CuNiMoTiOx, CuNiNbTiOx, CuNiTaTiOx, CuNiVTiOx, CuNiZnTiOx, FeCoAlTiOx, FeCoCrTiOx, FeCoMnTiOx, FeCoMoTiOx, FeCoNbTiOx, FeCoTaTiOx, FeCoVTiOx, FeCoZnTiOx, FeNiAlTiOx, FeNiCoTiOx, FeNiCrTiOx, FeNiMnTiOx, FeNiMoTiOx, FeNiNbTiOx, FeNiTaTiOx, FeNiVTiOx, FeNiZnTiOx, ZnCoAlTiOx, ZnCoCrTiOx, ZnCoMnTiOx, ZnCoMoTiOx, ZnCoNbTiOx, ZnCoTaTiOx, ZnCoVTiOx, ZnCoZnTiOx, ZnFeAlTiOx, ZnFeCrTiOx, ZnFeMnTiOx, ZnFeMoTiOx, ZnFeNbTiOx, ZnFeTaTiOx, ZnFeVTiOx, ZnFeZnTiOx, ZnNiAlTiOx, ZnNiCoTiOx, ZnNiCrTiOx, ZnNiMnTiOx, ZnNiMoTiOx, ZnNiNbTiOx, ZnNiTaTiOx, ZnNiVTiOx, and ZnNiZnTiOx.

According to still certain embodiments, the sensor material contains a carbon-based material selected from carbon black, activated carbon, microporous carbon, mesoporous carbon, micro-mesoporous carbon, pyrolytic carbon, carbon nanotube, carbon nanofiber, carbon nanospheres, carbon nanosheet, carbon nanowire, carbon nanorod, graphene, graphene oxide, and reduced graphene oxide.

In further embodiments, the carbon-based material has a dopant selected from sulfur, nitrogen, oxygen, boron, fluorine, bromine, iodine, chlorine, phosphorus, selenium, chlorine, and tellurium.

In still further embodiments, the carbon-based material is functionalized with one or more organic agents selected from amines, fatty acids, alcohols, thiols, aldehydes, phenols, esters, epoxy, polymers, silane coupling agents, carboxylic acids, nitriles, sulfonic acids, imides, isocyanates, ketones, amides, nucleic acids, amino acids, and mixtures thereof.

In some further embodiments, one of the one or more metals in the sensor material is a single-atom metal residing in a carbon framework.

In still some further embodiments, at least one of the one or more sensor materials contains one or more conjugated polymers selected from polypyrrole (PPy), polyaniline (PANI), polythiophene (PTh), poly(3,4-ethylenedioxythiophene) (PEDOT), poly(3-hexylthiophene) (P3HT), polyacetylene, polyphenylene vinylene (PPV), polyfluorene, polysulfone, polyindole, polyparaphenylene (PPP), and polycarbazole.

According to another aspect of the current disclosure, VOCs that can be detected using the sensor are selected form acetaldehyde, acetone, acetic acid, acetophenone, ammonia, benzaldehyde, butanal, butane, butanol, carbon dioxide (CO₂), carbon disulfide, capric acid, caproic acid, caprylic acid, chlorine, decane, dimethyl ether, dimethyl pyrazine, dimethyl sulfide, dimethyl trisulfide, ethane, ethanol, ethyl acetate, formaldehyde, hexane, heptane, hydrogen cyanide, hydrogen peroxide, hydrogen sulfide, indole, isobutyric acid, isoprene, isovaleric acid, lauric acid, linoleic acid, methane, methanol, methyl cyclohexane, methyl propanoate, methyl butanoate, methyl mercaptan, methyl thiocyanate, methyl thiolacetate, myristic acid, nitric oxide, nitrogen dioxide, nonane, octane, ozone, palmitic acid, pentafluoropropionamide, pentane, propane, propionic acid, propanol, stearic acid, sulfur dioxide, 2-aminoacetophenone, 2-butanol, 2-butanone, 2-ethylhexanol, 2-heptanone, 2-methoxy-5-methylthiophene, 2-methylbutanal, 2-methylbutanol, 2-methylbutyl 2-methylbutyrate, 2-methylbutyl isobutyrate, 2-methyl-3-(2-propenyl)-pyrazine, 2-nonanone, 2-pentene, 2-pentanol, 2-tridecenone, 3-(ethylthio)-propanal, 3-hydroxy-2-butanone, 3-methyl-1-butanol, 3-methylbutanal, 3-methylbutanoic acid, 3-methyl-1H-pyrrole, 6-methyl-5-hepten-2-one, 6-tridecane, 1,1,2,2-tetrachloroethane, 1-hydroxy-2-propanone, 1-Octanol, 1-undecane, dodecane, isobutyric acid, isopropyl acetate, and valeric acid.

According to a further aspect of the current disclosure, pathogens that generates VOCs include Acinetobacter anitratus, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Ehrlichia chaffeensis, Enterobacter cloacae, Enterococcus avium, Enterococcus durans, Enterococcus faecalis, Enterococcus faecium, Enterococcus galllinamm, Enterococcus maloratus, Escherichia coli, Francisella tularensis, Fusobacterium nucleatum, Enterobacter species, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumonia, Lactobacillus acidophilus, Lactobacillus bulgaricus, Lactobacillus casei, Lactobacillus delbrueckii, Lactococcus lactis, Legionella pneumophila, Listeria monocytogenes, Methanobacterium extroquens, Microbacterium multiforme, Micrococcus luteus, Moraxella catarrhalis, Morganella morganii, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Pasteurella multocida, Pasteurella tularensis, Porphyromonas gingivalis, Prevotella melaninogenica, Proteus vulgaris, Proteus mirabilis, Proteus penneri, Providencia stuartii, Pseudomonas aeruginosa, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Yersinia enterocolitica, Yersinia pestis and Yersinia pseudotuberculosis, and/or known to comprise one or more antibiotic-resistant strains descending from a known species, and/or known to comprise one or more extended spectrum beta-lactamase-producing strains descending from a known species, in particular the one or more extended spectrum beta-lactamase-producing strain is selected from the group consisting of: extended spectrum beta-lactamase-producing Escherichia coli, and extended spectrum beta-lactamase-producing Klebsiella pneumoniae.

According to still another embodiment, a sensor device has one or more sensors connected to a plurality of electrodes supported on a substrate to form an electrical conduit.

In some embodiments, the sensor device is configured to measure changes in resistance, conductance, alternating current (AC), frequency, capacitance, impedance, inductance, mobility, electrical potential, optical property or voltage threshold.

In other embodiments, the sensor device contains an array of sensors having 2 to 48 sensors. The sensors in the array are identical or different.

### BRIEF DESCRIPTION OF DRAWINGS

FIG. 1 shows TEM micrographs of a porous sensor material that is mesoporous Co₃O₄. FIG. 1 shows the TEM and SAED images (inset) of mesoporous Co₃O₄ materials synthesized using FDU-12 (panel a and panel b) and SBA-16 (panel c and panel d) templates, as well as the HRTEM.

FIG. 2A shows XRD patterns of materials obtained by calcining a mixture of cobalt nitrate and chromium nitrate at various temperatures.

FIG. 2B shows XRD patterns of materials obtained by calcining a mixture of cobalt nitrate and nickel nitrate at various temperatures.

FIG. 2C shows XRD patterns of materials by calcining a mixture of nickel nitrate and indium nitrate at various temperatures.

FIG. 2D shows XRD patterns of materials by calcining a mixture of nickel nitrate and aluminum nitrate at various temperatures.

FIG. 2E shows XRD patterns of materials by calcining a mixture of iron nitrate and indium nitrate at various temperatures.

FIG. 2F shows XRD patterns of materials by calcining a mixture of iron nitrate and manganese nitrate at various temperatures.

FIG. 3 shows the response signals of the sensor constructed with the porous Co₃O₄ material shown in FIG. 1 to exemplary VOCs.

FIG. 4A shows the response of the sensor constructed with the nanostructured CoOx to ethanol.

FIG. 4B shows the response of the sensor constructed with the nanostructured ZnO to CO₂.

FIG. 4C shows the response of the sensor constructed with the nanostructured LaCoSnOx to acetic acid.

FIG. 4D shows the response of the sensor constructed with the nanostructured InCoSnOx to benzene.

FIG. 4E shows the response of the sensor constructed with the nanostructured CoTbCuOx to 1-butanol.

FIG. 4F shows the response of the sensor constructed with the nanostructured and SnInFeCoOx to isoprene.

FIG. 5 is a flow chart describing a method for preparing a sensor material.

FIG. 6 shows the synthesized sensor material after heating and annealing.

FIG. 7 is an image of a sensor device having an array of 12 sensors integrated sensor array consisting of 12 individual sensors.

### DETAILED DESCRIPTION OF EMBODIMENTS

The methods, compositions, materials, sensors, and devices herein may be used for detecting and identifying wound infections using VOCs released from the pathogens of a subject. The infections occur to skin of palm, finger, ear, nose, face, eye, arm, leg, chest, breast, back, abdomen, and/or or foot. Sensors in conjugation with pattern recognition and machine-learning algorithms enable detection of wound infections.

The sensor contains one or more sensor materials. The sensor material can be non-porous or porous. The porous material can contain a single porous material or a mixture of porous materials. The porous material can be mesoporous, macroporous, microporous, nanoporous, or a hierarchical porous material, including mesoporous/macroporous hierarchical structure, microporous/macroporous hierarchical structure, microporous/mesoporous hierarchical structure, microporous/mesoporous/macroporous hierarchical structure, etc. The mesoporous structure is in a configuration selected from well-ordered mesoporous structure with regular pore arrangement, worm-like mesoporous structure with uniform pore size but without long-range regularity, or non-order mesostructure with pore size from 2-50 nm. The macroporous structure are a configuration selected from well-ordered macroporous structure or non-order macroporous structure with pore size from 50 nm to 50 μm. In further embodiment, the pore size of sensor materials is ranging from 0.4 to 2 nm, 2 to 50 nm, 50 nm to 200 nm, 200 nm to 500 nm, 500 nm to 1 μm, 1 to 50 μm. The specific surface area is 1-1000 m²/g.

The sensor material herein can also be in the form of a nanomaterial. Examples of nanomaterials include nanosphere, nanorods, nanowire, nanoporous, nanomesh, nanodot, nanostar, nanosheet, nanoplate, nanoflake, nanotube, nano-hollow sphere, nanocube, nanocage, nanopolyhedron, nanobelt, nanocylinder, nanohelix, nanoprism, nanoribbon, nanoring, nanotetrapod, nanodumbbell, nanocrescent, nanosculpture, nanobrush, nanofiber, nanocrystal, nanowhisker, nanoscroll, and nanocapsule, etc. Note that the sensor material can be a porous nanomaterial. Further, the sensor material comprises unary, binary, ternary, quaternary, quinary, senary, septenary, and octonary multiple-component metal oxides.

The elements in the sensor material may be selected from tin (Sn), terbium (Tb), cobalt (Co), zinc (Zn), indium (In), copper (Cu), nickel (Ni), chromium (Cr), manganese (Mn), tungsten (W), titanium (Ti), vanadium (V), iron (Fe), aluminum (Al), gallium (Ga), silver (Ag), gold (Au), palladium (Pd), rhodium (Rh), ruthenium (Ru), molybdenum (Mo), niobium (Nb), zirconium (Zr), yttrium (Y), lanthanum (La), platinum (Pt), silicon (Si), cerium (Ce), and tellurium (Te). Some of the examples of the sensor material include CoOx, SnOx, ZnOx, NiOx, VOx, FeOx, CuOx, CrOx, InOx, TbOx, MnOx, WOx, TiOx, AlOx, GaOx, AgOx, AuOx, PdOx, RhOx, RuOx, MoOx, NbOx, ZrOx, YOx, LaOx, PtOx, SiOx, CeOx, TeOx, AgAlOx, AgAuOx, AgCoOx, AgCrOx, AgCuOx, AgFeOx, AgGaOx, AgInOx, AgMnOx, AgMoOx, AgNiOx, AgPdOx, AgPtOx, AgRhOx, AgRuOx, AgTiOx, AgVOx, AgWOx, AgZnOx, AgZrOx, AlCoOx, AlCrOx, AlCuOx, AlFeOx, AlGaOx, AlInOx, AlMnOx, AlMoOx, AlNiOx, AlPdOx, AlPtOx, AlRhOx, AlRuOx, AlTiOx, AlVOx, AlWOx, AlZnOx, AlZrOx, AuCoOx, AuCrOx, AuCuOx, AuFeOx, AuGaOx, AuInOx, AuMnOx, AuMoOx, AuNiOx, AuPdOx, AuPtOx, AuRhOx, AuRuOx, AuTiOx, AuVOx, AuWOx, AuZnOx, AuZrOx, CoCrOx, CoCuOx, CoFeOx, CoGaOx, CoInOx, CoMnOx, CoMoOx, CoNiOx, CoPdOx, CoPtOx, CoRhOx, CoRuOx, CoTiOx, CoVOx, CoWOx, CoZnOx, CoZrOx, CrCuOx, CrFeOx, CrGaOx, CrInOx, CrMnOx, CrMoOx, CrNiOx, CrPdOx, CrPtOx, CrRhOx, CrRuOx, CrTiOx, CrVOx, CrWOx, CrZnOx, CrZrOx, CuFeOx, CuGaOx, CuInOx, CuMnOx, CuMoOx, CuNiOx, CuPdOx, CuPtOx, CuRhOx, CuRuOx, CuTiOx, CuVOx, CuWOx, CuZnOx, CuZrOx, FeGaOx, FeInOx, FeMnOx, FeMoOx, FeNiOx, FePdOx, FePtOx, FeRhOx, FeRuOx, FeTiOx, FeVOx, FeWOx, FeZnOx, FeZrOx, GaInOx, GaMnOx, GaMoOx, GaNiOx, GaPdOx, GaPtOx, GaRhOx, GaRuOx, GaTiOx, GaVOx, GaWOx, GaZnOx, GaZrOx, InMnOx, InMoOx, InNiOx, InPdOx, InPtOx, InRhOx, InRuOx, AlFeTiOx, AlGaFeOx, AlGaMnOx, AlGaTiOx, AlInZnOx, AlNiZnOx, AlTiVxOx, AlVZnOx, AlZnCuOx, AlZnFeOx, AlZnMnOx, AlZnNiOx, AlZnTiOx, CoCrFeOx, CoCrMnOx, CoCrNiOx, CoCrTiOx, CoFeTiOx, CoMnNiOx, CoMnTiOx, CoNiTiOx, CoTiVxOx, CoTiZrOx, CoVZnOx, CrFeTiOx, CrMnTiOx, CrNiTiOx, FeMnTiOx, FeTiVxOx, FeTiZrOx, FeVZnOx, GaInZnOx, GaMnTiOx, GaTiZrOx, GaVZnOx, InMnZnOx, InNiZnOx, InTiZrOx, InVZnOx, MnNiZnOx, MnTiVxOx, MnTiZrOx, MnVZnOx, NiTiVxOx, NiTiZrOx, NiVZnOx, TiVZrOx, TiZrZnOx, TiZrCuOx, TiZrNiOx, TiZrCoOx, TiZrMnOx, TiZrFeOx, TiZrAlOx, TiZnCuOx, TiZnFeOx, TiZnMnOx, TiZnNiOx, TiZnCoOx, TiZnCrOx, TiZnAlOx, VZrZnOx, VZrCuOx, VZrCrOx, VZrMnOx, VZrNiOx, VZrCoOx, VZrFeOx, VZrAlOx, YAlOx, YCrOx, YFeOx, YInOx, YNiOx, YTiOx, ZnCrTiOx, ZnFeTiOx, ZnMnTiOx, ZnNiTiOx, ZnTiVxOx, ZnTiZrOx, ZrCrFeOx, ZrCrMnOx, ZrCrNiOx, ZrCrTiOx, ZrFeTiOx, ZrMnNiOx, ZrMnTiOx, ZrNiTiOx, ZrTiVxOx, ZrTiZnOx, ZrVZnOx, ZrZnCuOx, ZrZnFeOx, ZrZnMnOx, ZrZnNiOx, ZrZnCoOx, ZrZnAlOx, AlCoCrTiOx, AlCoMnTiOx, AlCoNbTiOx, AlCoTaTiOx, AlCoVTiOx, AlFeCoTiOx, AlFeCrTiOx, AlFeMnTiOx, AlFeMoTiOx, AlFeNbTiOx, AlFeTaTiOx, AlFeVTiOx, AlFeZnTiOx, AlNiCoTiOx, AlNiCrTiOx, AlNiMnTiOx, AlNiMoTiOx, AlNiNbTiOx, AlNiTaTiOx, AlNiVTiOx, AlNiZnTiOx, CuCoAlTiOx, CuCoCrTiOx, CuCoMnTiOx, CuCoMoTiOx, CuCoNbTiOx, CuCoTaTiOx, CuCoVTiOx, CuCoZnTiOx, CuFeAlTiOx, CuFeCrTiOx, CuFeMnTiOx, CuFeMoTiOx, CuFeNbTiOx, CuFeTaTiOx, CuFeVTiOx, CuFeZnTiOx, CuNiAlTiOx, CuNiCoTiOx, CuNiCrTiOx, CuNiMnTiOx, CuNiMoTiOx, CuNiNbTiOx, CuNiTaTiOx, CuNiVTiOx, CuNiZnTiOx, FeCoAlTiOx, FeCoCrTiOx, FeCoMnTiOx, FeCoMoTiOx, FeCoNbTiOx, FeCoTaTiOx, FeCoVTiOx, FeCoZnTiOx, FeNiAlTiOx, FeNiCoTiOx, FeNiCrTiOx, FeNiMnTiOx, FeNiMoTiOx, FeNiNbTiOx, FeNiTaTiOx, FeNiVTiOx, FeNiZnTiOx, ZnCoAlTiOx, ZnCoCrTiOx, ZnCoMnTiOx, ZnCoMoTiOx, ZnCoNbTiOx, ZnCoTaTiOx, ZnCoVTiOx, ZnCoZnTiOx, ZnFeAlTiOx, ZnFeCrTiOx, ZnFeMnTiOx, ZnFeMoTiOx, ZnFeNbTiOx, ZnFeTaTiOx, ZnFeVTiOx, ZnFeZnTiOx, ZnNiAlTiOx, ZnNiCoTiOx, ZnNiCrTiOx, ZnNiMnTiOx, ZnNiMoTiOx, ZnNiNbTiOx, ZnNiTaTiOx, ZnNiVTiOx, ZnNiZnTiOx. Used in this disclosure, a chemical formula ending with an “x” represents that all compositions that have the same elements in the chemical formula but the amounts of such elements vary in the compositions.

These formulae reflect the elements in the sensor material while the concentration of each element may vary from, e.g., 0.01 to 0.99, 0.1 to 0.9, 0.2 to 0.8, 0.3 to 0.7, or 0.4 to 0.6.

In some embodiments, the sensor material comprises unary, binary, ternary, quaternary, quinary, and senary single or multiple-component elementary metal particles. The metal can be one or more amongst aluminum (Al), antimony (Sb), bismuth (Bi), boron (B), cerium (Ce), chromium (Cr), cobalt (Co), copper (Cu), gold (Au), indium (In), iridium (Ir), iron (Fe), lanthanum (La), lead (Pb), manganese (Mn), molybdenum (Mo), nickel (Ni), niobium (Nb), osmium (Os), palladium (Pd), platinum (Pt), rhodium (Rh), ruthenium (Ru), selenium (Se), silicon (Si), silver (Ag), tantalum (Ta), tellurium (Te), terbium (Tb), tin (Sn), titanium (Ti), tungsten (W), vanadium (V), ytterbium (Yb), yttrium (Y), zinc (Zn), zirconium (Zr). The size of the particles is ranging from 0.5 nm to 500 nm, 1 nm to 100 nm, 10 nm to 200 nm, or 1 nm to 10 nm, 10 nm to 30 nm, 30 nm to 50 nm, 50 nm to 100 nm, 100 nm to 500 nm. The shape of the particles can be nanosphere, nanorods, nanowire, nanodot, nanoporous, nanomesh, nanostar, nanosheet, nanoplate, nanoflake, nanotube, nano-hollow sphere, nanocube, nanocage, nanopolyhedron, nanobelt, nanocylinder, nanohelix, nanoprism, nanoribbon, nanoring, nanotetrapod, nanodumbbell, nanocrescent, nanosculpture, nanobrush, nanofiber, nanocrystal, nanowhisker, nanoscroll, and nanocapsule. Some examples are listed as follows: Pt nanospheres, Au nanodots, Ag nanowires, Ag—Au nanocubes, Os nanorods, Fe nanoparticles, Pd nanowire, Pt—Ru nanocube, Pt—Ru nanocrystal, Ru nanocage, etc.

In some embodiments, the sensor material may further contain carbon-based material. The carbon-based material may be carbon black, active carbon, microporous carbon, mesoporous carbon, micro-mesoporous carbon, pyrolytic carbon, carbon nanotube, carbon nanofiber, carbon nanospheres, carbon nanosheet, carbon nanowire, carbon nanorod, graphene, graphene oxide and reduced graphene oxide. The carbon-based material may be doped with sulfur, nitrogen, oxygen, boron, fluorine, bromine, iodine, chlorine, phosphorus, selenium, chlorine, tellurium, etc.

In some embodiments, the sensor material may contain carbon material that functionalized with organic agents, such as amines, fatty acids, alcohols, thiols, aldehydes, phenols, esters, epoxy, polymers, silane coupling agents, carboxylic acids, nitriles, sulfonic acids, imides, isocyanates, ketones, amides, nucleic acids, amino acids, or mixtures thereof.

In some embodiments, the sensor material may contain carbon material with single-atom metal in its carbon framework. The single-atom metal can be aluminum (Al), antimony (Sb), bismuth (Bi), boron (B), carbon (C), cerium (Ce), chromium (Cr), cobalt (Co), copper (Cu), gold (Au), indium (In), iridium (Ir), iron (Fe), lanthanum (La), lead (Pb), manganese (Mn), molybdenum (Mo), nickel (Ni), niobium (Nb), osmium (Os), palladium (Pd), platinum (Pt), rhodium (Rh), ruthenium (Ru), selenium (Se), silicon (Si), silver (Ag), tantalum (Ta), tellurium (Te), terbium (Tb), tin (Sn), titanium (Ti), tungsten (W), vanadium (V), ytterbium (Yb), yttrium (Y), zinc (Zn), zirconium (Zr).

In some embodiments, the sensor material may be a composite that contains both elemental metal and metal oxide as components to form composite sensor materials, with metal nanoparticles and metal oxides described and listed.

In some embodiments, the sensor material may be a composite that contains carbon-based material, metal nanoparticles and metal oxides.

In some embodiments, the sensor material may contain one or more conjugated polymers, such as polypyrrole (PPy) materials, polyaniline (PANI) materials, polythiophene (PTh) materials, poly(3,4-ethylenedioxythiophene) (PEDOT) materials, poly(3-hexylthiophene) (P3HT) materials, polyacetylene materials, polyphenylene vinylene (PPV) materials, polyfluorene materials, polysulfone materials, polyindole materials, polyparaphenylene (PPP) materials, and polycarbazole materials, etc. In some embodiments the conjugated polymer is mixed with the carbon-based material.

The sensor material can be made into a structure, e.g., a patch of material supported on a substrate. Multiple structures made of the sensor material form a sensor array. The sensor array has two or three or four or eight or twelve or more sensors, which may be used for detecting one or more gases from metabolite gas mixtures emanated from pathogens of wound infection.

In some embodiments, the sensor contains a substrate that support the sensor material and a plurality of electrodes on said substrate that form an electrical conduit.

In some embodiments, the sensor can be a capacitive sensor, a resistive sensor, a chemiresistive sensor, an impedance sensor, and a field effect transistor sensor. Each possibility represents a separate embodiment of the present disclosure. In exemplary embodiments, the sensor is configured as a chemiresistive sensor.

In certain embodiments, the sensor further comprises a detection means comprising a device for measuring changes in resistance, conductance, alternating current (AC), frequency, capacitance, impedance, inductance, mobility, electrical potential, optical property or voltage threshold. Each possibility represents a separate embodiment of the present disclosure.

In particular embodiments, the present disclosure provides a sensor array for diagnosing wound infection caused by pathogens in a subject, the sensor array having a plurality of sensors, for example between 2 and 6 and 8 and 12 and 32 and 48 sensors consisting essentially of at least two of sensor materials, a substrate, a plurality of electrodes on said substrate, and a detection means.

The sensors in the array can be formed into a variety of shapes and sizes, such as a patch of material supported on a substrate.

In certain embodiments, the sensor array can be used to identify the type of pathogen causing the wound infection based on the pattern of VOCs detected by the sensors. The pattern can be analyzed using pattern recognition and machine-learning algorithms known in the art to classify the VOCs and to identify the type of pathogen causing the infection.

In certain embodiments, the sensor device may be in contact or not in contact with a subject's wound, and upon exposing to at least one VOC indicative of wound infection caused by pathogens such as Staphylococcus epidermidis, Streptococcus pyogenes, Enterococcus faecium, Staphylococcus aureus, Klebsiella pneumonia, Acinetobacter baumannii, Pseudomonas aeruginosa, Enterobacter species, Escherichia coli, Proteus mirabilis, Serratia marcescens, Enterobacter cloacae, and Acinetobacter anitratus, Lactobacillus delbrueckii, Gardnerella vaginalis, antibiotic-resistant strains, and other pathogens associated with wound infections sensor material the electrical conductivity between the electrodes changes thereby providing a measurable signal indicative of wound infection. The signal can be detected by a detection means comprising a device for measuring changes in resistance, conductance, alternating current (AC), frequency, capacitance, impedance, inductance, mobility, electrical potential, optical property or voltage threshold.

The pathogens of wound infection comprise one or more bacterial or fungi species, but not limited from Acinetobacter anitratus, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Ehrlichia chaffeensis, Enterobacter cloacae, Enterococcus avium, Enterococcus durans, Enterococcus faecalis, Enterococcus faecium, Enterococcus galllinamm, Enterococcus maloratus, Escherichia coli, Francisella tularensis, Fusobacterium nucleatum, Enterobacter species, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumonia, Lactobacillus acidophilus, Lactobacillus bulgaricus, Lactobacillus casei, Lactobacillus delbrueckii, Lactococcus lactis, Legionella pneumophila, Listeria monocytogenes, Methanobacterium extroquens, Microbacterium multiforme, Micrococcus luteus, Moraxella catarrhalis, Morganella morganii, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Pasteurella multocida, Pasteurella tularensis, Porphyromonas gingivalis, Prevotella melaninogenica, Proteus vulgaris, Proteus mirabilis, Proteus penneri, Providencia stuartii, Pseudomonas aeruginosa, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Yersinia enterocolitica, Yersinia pestis and Yersinia pseudotuberculosis, and/or known to comprise one or more antibiotic-resistant strains descending from a known species, and/or known to comprise one or more extended spectrum beta-lactamase-producing strains descending from a known species, in particular the one or more extended spectrum beta-lactamase-producing strain is selected from the group consisting of: extended spectrum beta-lactamase-producing Escherichia coli, and extended spectrum beta-lactamase-producing Klebsiella pneumoniae.

Antibiotic-resistant bacterial strains are selected from the group consisting of: Carbapanem-resistant Acinetobacter baumannii, carbapanem-resistant Pseudomonas aeruginosa, vancomycin-resistant Enterococcus faecium, methicillin-resistant Staphylococcus aureus, vancomycin-resistant Staphylococcus aureus, clarithromycin-resistant Helicobacter pylori, fluoroquinolone-resistant Campylobacter coli, fluoroquinolone-resistant Campylobacter fetus, fluoroquinolone-resistant Campylobacter jejuni, fluoroquinolone-resistant Campylobacter pylori, fluoroquinolone-resistant Salmonella enteritidis, fluoroquinolone-resistant Salmonella typhi, fluoroquinolone-resistant Salmonella typhimurium, cephalosporin-resistant Neisseria gonorrhoeae, fluoroquinolone-resistant Neisseria gonorrhoeae, penicillin-non-susceptible Streptococcus pneumonia, ampicillin-resistant Haemophilus influenza, fluoroquinolone-resistant Shigella dysenteriae, carbapanem-resistant Escherichia coli, carbapanem-resistant Klebsiella pneumonia, carbapanem-resistant Enterobacter cloacae, carbapanem-resistant Serratia marcescens, carbapanem-resistant Proteus vulgaris, carbapanem-resistant Proteus mirabilis, carbapanem-resistant Proteus penneri, carbapanem-resistant Providencia stuartii, carbapanem-resistant Morganella morganii, cephalosporin-resistant Escherichia coli, cephalosporin-resistant Klebsiella pneumonia, cephalosporin-resistant Enterobacter cloacae, cephalosporin-resistant Serratia marcescens, cephalosporin-resistant Proteus vulgaris, cephalosporin-resistant Proteus mirabilis, cephalosporin-resistant Proteus penneri, cephalosporin-resistant Providencia stuartii and cephalosporin-resistant Morganella morganii.

In certain embodiments, volatile organic compounds (VOCs) include a diverse group of carbon-based molecules, including Acetaldehyde, Acetone, Acetic acid, Acetophenone, Ammonia, Benzaldehyde, Butanal, Butane, Butanol, Carbon dioxide (CO₂), Carbon disulfide, Capric acid, Caproic acid, Caprylic acid, Chlorine, Decane, Dimethylether, Dimethyl pyrazine, Dimethyl sulfide, Dimethyl trisulfide, Ethane, Ethanol, Ethyl acetate, Formaldehyde, Hexane, Heptane, Hydrogen cyanide, Hydrogen peroxide, Hydrogen sulfide (H2S), Indole, Isobutyric acid, Isoprene, Isovaleric acid, Lauric acid, Linoleic acid, Methane, Methanol, Methyl cyclohexane, Methyl propanoate, Methyl butanoate, Methyl mercaptan, Methyl thiocyanate, Methyl thiolacetate, Myristic acid, Nitric oxide, Nitrogen dioxide, Nonane, Octane, Ozone, Palmitic acid, Pentafluoropropionamide, Pentane, Propane, Propionic acid, Propanol, Stearic acid, Sulfur dioxide, 2-aminoacetophenone, 2-butanol, 2-butanone, 2-ethylhexanol, 2-heptanone, 2-methoxy-5-methylthiophene, 2-methylbutanal, 2-methylbutanol, 2-methylbutyl 2-methylbutyrate, 2-methylbutyl isobutyrate, 2-methyl-3-(2-propenyl)-pyrazine, 2-nonanone, 2-pentene, 2-pentanol, 2-tridecenone, 3-(ethylthio)-propanal, 3-hydroxy-2-butanone, 3-methyl-1-butanol, 3-methylbutanal, 3-methylbutanoic acid, 3-methyl-1H-pyrrole, 6-methyl-5-hepten-2-one, 6-tridecane, 1,1,2,2-tetrachloroethane, 1-hydroxy-2-propanone, 1-Octanol, 1-undecane, Dodecane, Isobutyric acid, Isopropyl acetate, Methane, Valeric acid, which are volatile at ambient temperatures. VOC detection has the advantage of being painless, non-invasive and reproducible.

The present invention provides a sensor material and sensor device for detecting VOCs emitted from an infectious wound for the purpose of wound infection detection. The sensor device has a substrate and a plurality of electrodes on the substrate that form an electrical conduit. The sensor is supported on the substrate and comprises one or more sensor materials, which may be non-porous or porous, and can be in the form of a nanomaterial. The porous material can be mesoporous, macroporous, microporous, nanoporous, or a hierarchical porous material. The sensor material may also contain carbon-based material and/or single-atom metal in its carbon framework. The sensor material may further contain conjugated polymers, such as polypyrrole (PPy) materials, polyaniline (PANI) materials, polythiophene (PTh) materials, poly(3,4-ethylenedioxythiophene) (PEDOT) materials, poly(3-hexylthiophene) (P3HT) materials, polyacetylene materials, polyphenylene vinylene (PPV) materials, polyfluorene materials, polysulfone materials, polyindole materials, polyparaphenylene (PPP) materials, polycarbazole materials, or mixtures thereof.

The sensor device can be a capacitive sensor, a resistive sensor, a chemiresistive sensor, an impedance sensor, or a field effect transistor sensor, and may include a detection means comprising a device for measuring changes in resistance, conductance, AC, frequency, capacitance, impedance, inductance, mobility, electrical potential, optical property, or voltage threshold. The sensor device can be configured as a sensor array having multiple sensors for detecting one or more gases from metabolite gas mixtures emanated from pathogens of wound infection.

The substrate that supports the sensor material and the plurality of electrodes can be made of any suitable material, such as silicon, glass, plastic, or ceramic. The electrodes can be made of any suitable conductive material, such as gold, silver, platinum, or carbon. The detection means can comprise a device for measuring changes in resistance, conductance, alternating current (AC), frequency, capacitance, impedance, inductance, mobility, electrical potential, optical property or voltage threshold. In certain embodiments, the sensor is configured as a chemiresistive sensor.

In another embodiment, the present disclosure provides a method for preparing a sensor device. The method comprises depositing a sensor material onto a substrate, such as a silicon substrate or other suitable substrate, using any deposition method known in the art, such as spin-coating, dip-coating, drop-casting, inkjet printing, or any other method known in the art. The deposition can be performed on a single electrode or a plurality of electrodes, depending on the desired configuration of the sensor device. The sensor device can be a single sensor or a sensor array, depending on the number of sensor materials and electrodes used.

The sensor array signals detected is obtained in response to the changes of electrical resistances of sensor materials.

The methods herein may comprise exposing the sensor array to gas mixtures emanated from pathogens of wound infection. As used herein, gas mixtures may comprise VOCs or vapor from a subject, e.g., from the skin or breath of a subject. In some cases, the VOCs or vapor is emitted from the skin of a subject. The skin may be that of any wound part of the subject, e.g., the palm, finger, arm, leg, back, abdomen, or foot of the subject. In some examples, the gas mixtures comprise diverse odor of chemical classes, such as aldehydes, alcohols, ketones, acids, Sulphur containing compounds, esters, hydrocarbons and nitrogen containing compounds.

The methods may be performed at a relatively low operation temperature. In some embodiments, the operation temperature may be at most 250° C., at most 200° C., at most 150° C., at most 100° C., at most 80° C., at most 60° C., at most 50° C., at most 40° C., at most 30° C., at most 20° C., or at most 10° C. In some examples, the operation temperature may be in a range from about −30° C. to about 40° C., e.g., from about 0° C. to 30° C., from about 10° C. to about 30° C., or from about 20° C. to about 25° C. In some examples, the operation temperature may be 50° C., or less.

The methods, gas sensors, and devices herein may detect relatively levels of gas mixtures. In some cases, the methods and devices may be capable of detecting VOCs at a concentration of 5000 parts per million (ppm) or less, 4000 ppm or less, 3000 ppm or less, 2000 ppm or less, 1000 ppm or less, 500 ppm or less, 250 ppm or less, 100 ppm or less, 50 ppm or less, 10 ppm or less, 1 ppm or less, 800 parts per billion (ppb) or less, 600 ppb or less, 500 ppb or less, 400 ppb or less, 200 ppb or less, 100 ppb or less, 80 ppb or less, 60 ppb or less, 40 ppb or less, 20 ppb or less, 10 ppb or less, or 1 ppb or less, of gases in gas mixtures. In some cases, the methods, sensors, and devices may be configured to have a limit of detection of 5000 ppm or less of gases in gas mixtures. By “limit of detection” is meant the lowest quantity of a substance that can be distinguished from the absence of that substance (e.g., a blank value). In certain cases, the gas sensor or device are configured to have a limit of detection of 1000 ppm or less, 500 ppm or less, such as 400 ppm or less, including 300 ppm or less, 200 ppm or less, 100 ppm or less, 75 ppm or less, 50 ppm or less, 25 ppm or less, 20 ppm or less, 15 ppm or less, 10 ppm or less, 5 ppm or less, 1 ppm or less, 500 ppb or less, 100 ppb or less, 50 ppb or less, 10 ppb or less, or 1 ppb or less. In certain cases, the gas sensor or device is configured to have a limit of detection of 1 ppm or less. In certain cases, the gas sensor or device is configured to detect at least 1 ppb, at least 10 ppb, at least 50 ppb, at least 100 ppb, at least 500 ppb, at least 1 ppm, at least 5 ppm, at least 10 ppm, at least 15, ppm, at least 20 ppm, at least 25 ppm, at least 50 ppm, at least 75 ppm, at least 100 ppm, or at least 200 ppm of the VOCs.

The method according to any one of the previous items, wherein detecting the set of sensor signals comprises sensor array both with the gas mixtures having the volatile compounds contained or accumulated therein and with gas mixtures not having the volatile compounds contained or accumulated therein.

The subject may relate to an animal, including mammals, prefer humans, to which the method of the present disclosure may be applied. Mammalian species that can benefit from the disclosure include, but are not limit to apes. Chimpanzees, orangutans, humans, monkeys; domesticated animals (e.g. pets) such as dogs, cats, guinea pigs, hamsters; large domesticated animals such as cattle, horses, goats, sheep, where the use of the disclosure for veteran purposes be mentioned. Also, a subject could be any wild animal, as the disclosure could be used both for veteran and tracking purposes.

Identification of bacteria may comprise a step of volatile organic or inorganic compounds obtained from pathogens of wound infection in a subject with a sensor array. A sensor array will be any device capable of generating an electrical signal changing in response to interaction with volatile compounds of interest. The sensor signals may be any observable change in one or more quantifiable entities such as resistance, voltage, frequency, and the like.

The reference signals comprise known pathogens, such as bacteria and fungi. The reference signals could be established using standard samples obtained from in vitro grown pathogens, or in vivo infected and non-infected patients or animals that are assessed both by the method of the present disclosure and/or by conventional means to identify pathogens found therein.

### Embodiments

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

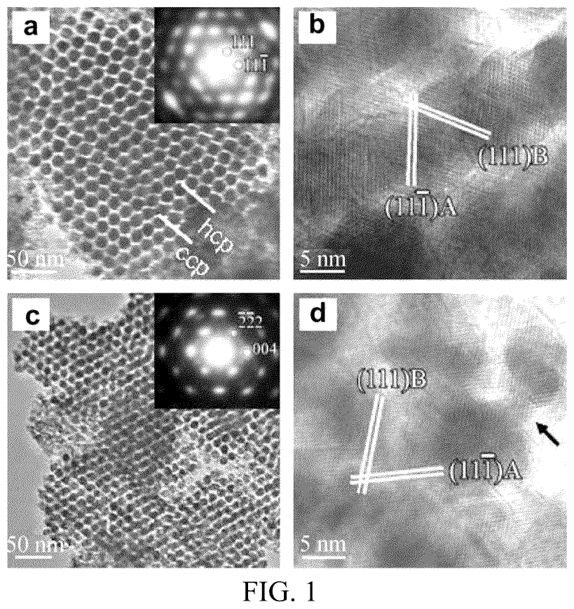

FIG. 1 shows TEM micrographs of a porous sensor material that is made of Co₃O₄. TM micrographs indicate a mesoporous structure having a relatively uniform pore size of about 15 nm. The mesoporous Co₃O₄ in panels a and b was synthesized using a FDU-12 template while the one in panels c and d was synthesized using a SBA-16 template.

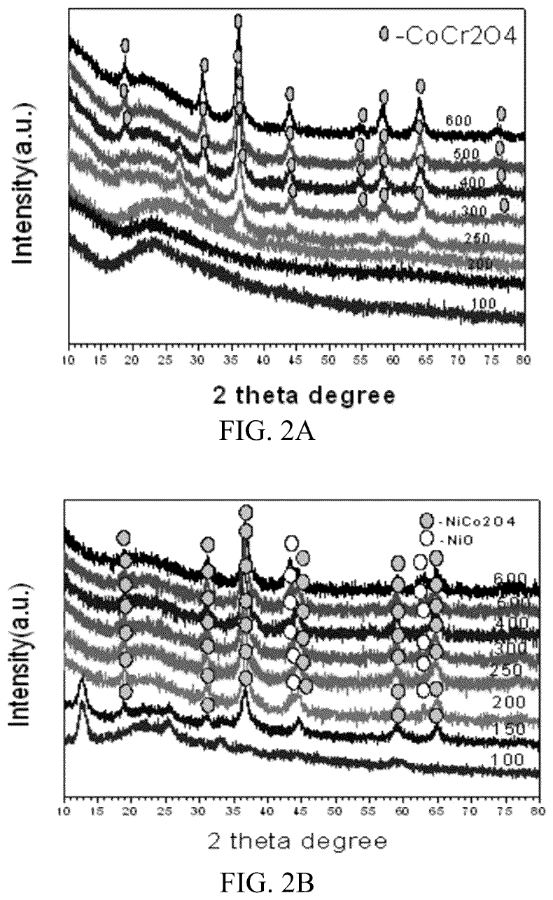

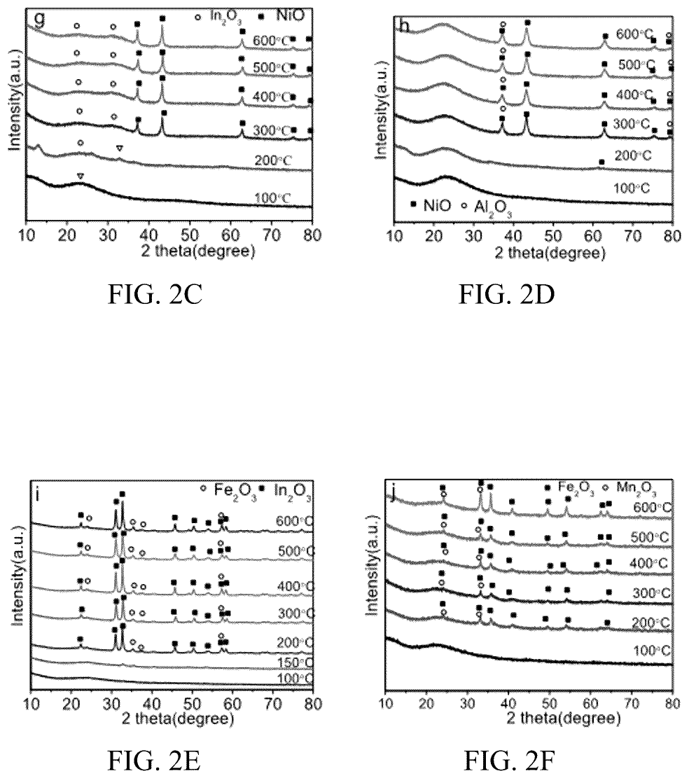

FIG. 2A shows a series of XRD patterns of materials obtained by calcining a mixture of cobalt nitrate and chromium nitrate at 100° C.-600° C. Heating or calcination at a temperature below 200° C. does not appear to produce any crystalline structure, while calcination at a temperature of 400° C. or higher results in stronger diffraction peaks, indicating the formation of crystalline CoCr₂O₄ with higher crystallinity.

FIG. 2B shows a series of XRD patterns obtained by heating a mixture of cobalt nitrate and nickel nitrate precursors at 100° C.-600° C. XRD peaks attributable to NiCo₂O₄ emerge at 150° C. while XRD peaks attributable to Ni emerge above 200° C.

FIG. 2C shows a series of XRD patterns obtained by heating a mixture of nickel nitrate and indium nitrate at 100° C.-600° C. XRD peaks attributable to In₂O₃ or NiO start to emerge at 300° C. while XRD peaks for NiO are more distinct than those for In₂O₃.

Likewise, FIG. 2D shows XRD patterns of materials by calcining a mixture of nickel nitrate and aluminum nitrate at 100° C.-600° C. XRD peaks attributable to Al₂O₃ or NiO start to emerge at 300° C. while XRD peaks attributable to NiO is more distinct.

FIG. 2E shows XRD patterns of materials by calcining a mixture of iron nitrate and indium nitrate at 100° C.-600° C. XRD peaks attributable to Fe₂O₃ or In₂O₃ start to emerge at 200° C. and grow stay relatively the same up to 600° C., indicating more complete crystallization at about 200° C.

FIG. 2F shows XRD patterns of materials by calcining a mixture of iron nitrate and manganese nitrate at various temperatures.

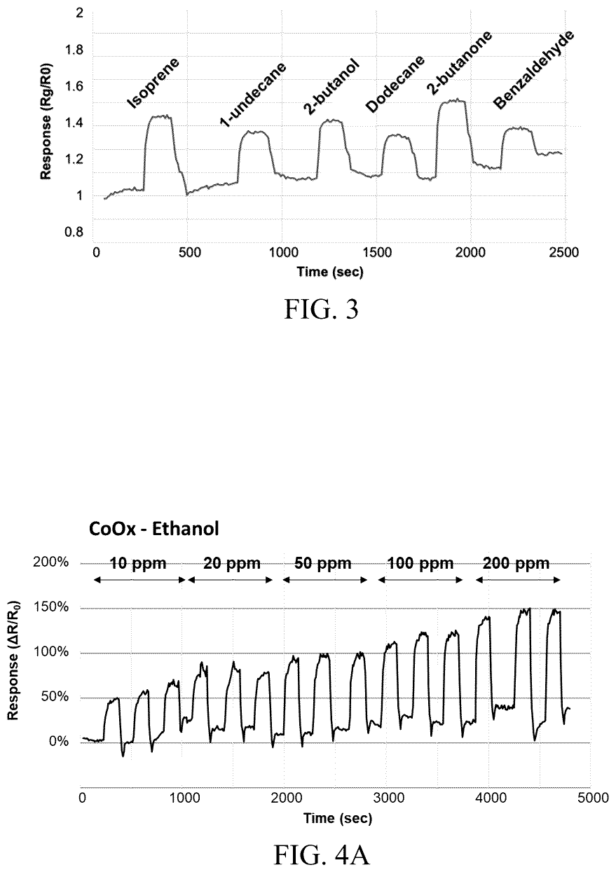

FIG. 3 exhibits the response of the sensor constructed with the porous Co₃O₄ material shown in FIG. 1 to various VOCs, expressed as R_{g}/R₀. R_{g} represents the sensor resistance in the presence of a gas, while R₀ is the sensor resistance in clean air. The porous Co₃O₄ exhibits a clear response to gas pulses that contain isoprene, 1-undecene, 2-butanol, dodecane, 2-butanone, or benzaldehyde, suggesting its potential as a sensitive detector for these VOCs. The results demonstrate the effectiveness of the Co₃O₄ material as a sensing platform for detecting a range of VOCs.

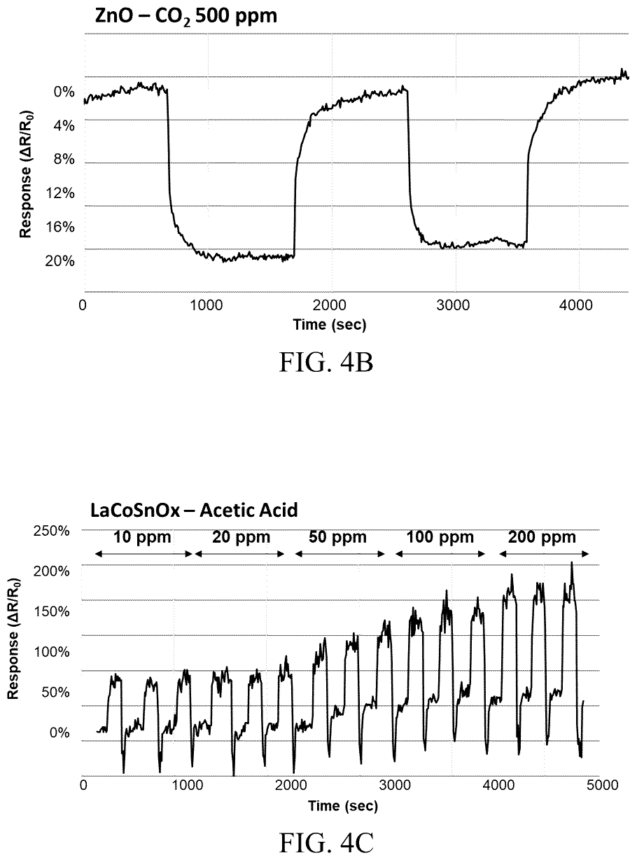

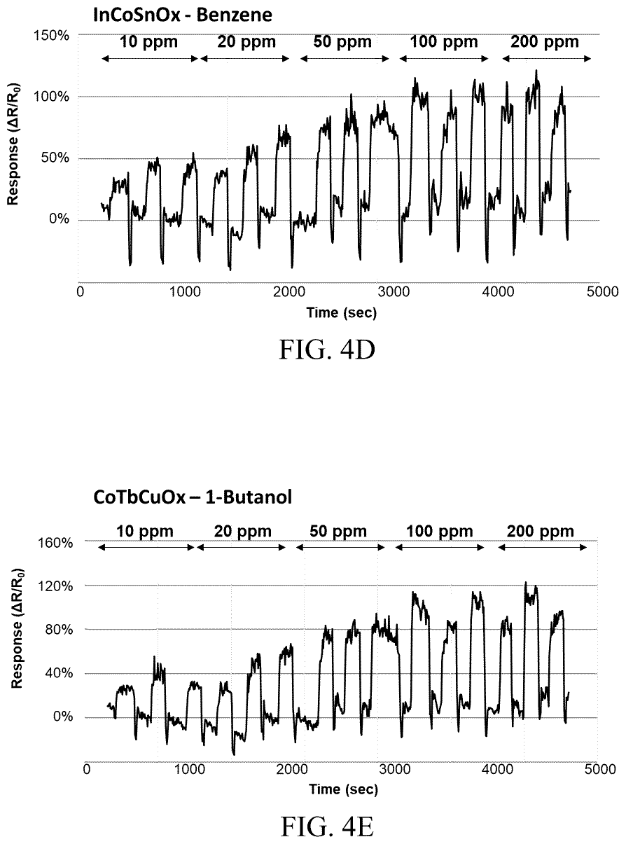

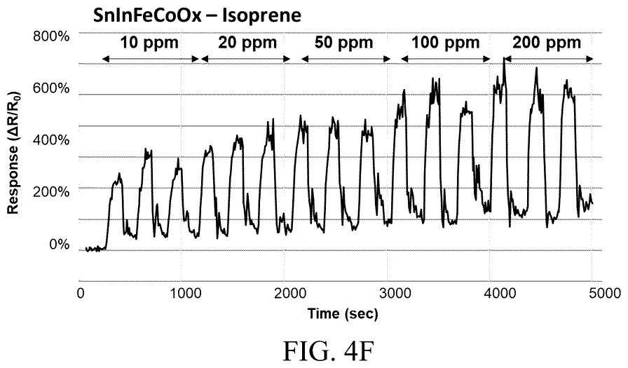

FIG. 4A shows the response of the sensor constructed with the nanostructured CoOx to pulses of ethanol-containing gas (i.e., ethanol pulses) from about 1 to 20 ppm. The response is shown as the percentage of ΔR (R_{g}−R₀) over R₀. FIG. 4B shows the response of the sensor constructed with the nanostructured ZnO to pulses of 500 ppm CO₂. FIG. 4C shows the response of the sensor constructed with the nanostructured LaCoSnOx to acetic acid pluses from 100 ppb to 2 ppm. FIG. 4D shows the response of the sensor constructed with the nanostructured InCoSnOx to benzene pulses from 10 ppm to 200 ppm. FIG. 4E shows the response of the sensor constructed with the nanostructured CoTbCuOx to 1-butano pulses from 1 ppm to 20 ppm. FIG. 4F shows the response of the sensor constructed with the nanostructured and SnInFeCoOx to isoprene pulses from 10 ppm to 200 ppm. Used herein, “nanostructured” means that the features of the material, e.g., pore size, particle size, are on the nanoscale.

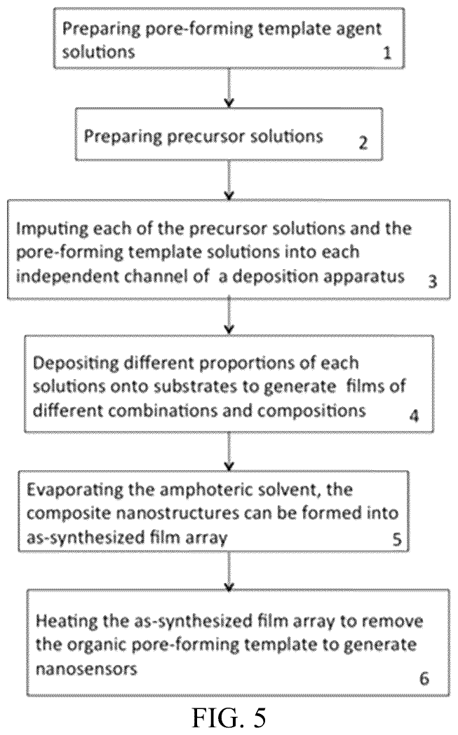

FIG. 5 describes an exemplary method of preparing a sensor. In the first two steps, a template solution that contains a pore-forming template agent (e.g., FDU-12, SBA-16, etc.) is prepared and one or more precursor solutions each containing a precursor of the sensor material is/are also prepared. The template solution and the precursor solutions are each put into its designated channel in a deposition apparatus. Different solutions are metered according to their proportion on a substrate to form one or more films having the same or different compositions. The films are heated to evaporate the liquid, leaving a film array on the substrate. The film array is annealed, i.e., heated to a prescribed temperature so that the organic pore-forming template is disassociated and removed. The resulting film is an array of sensor material films on the substrate to form a package, e.g., a chip. The package is then integrated into a device having necessary electronic components to form the sensor device.

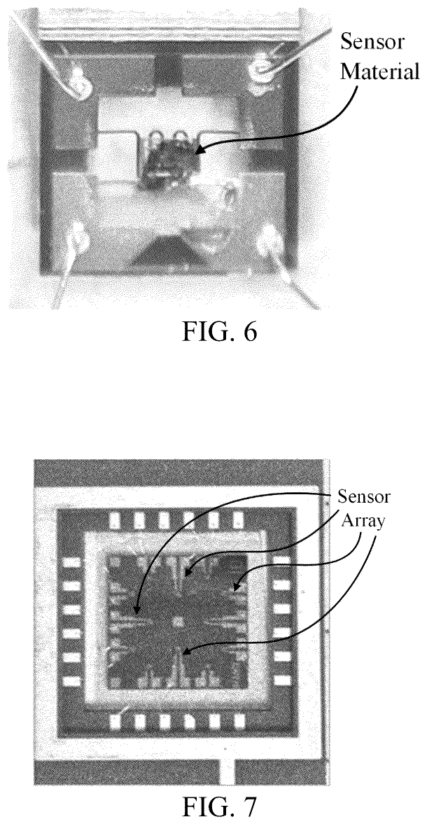

FIG. 6 shows a highly sensitive sensor material patch formed on a substrate.

FIG. 7 shows an array of 12 sensors in the middle portion of a chip, three sensors on each side. Each sensor is connected to two wire bonding pads arranged on the periphery of the chip.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A sensor comprising one or more sensor materials, each sensor material containing one or more metals selected from tin (Sn), terbium (Tb), cobalt (Co), zinc (Zn), indium (In), copper (Cu), nickel (Ni), chromium (Cr), manganese (Mn), tungsten (W), titanium (Ti), vanadium (V), iron (Fe), aluminum (Al), gallium (Ga), silver (Ag), gold (Au), palladium (Pd), rhodium (Rh), ruthenium (Ru), molybdenum (Mo), niobium (Nb), zirconium (Zr), yttrium (Y), lanthanum (La), platinum (Pt), silicon (Si), cerium (Ce), and tellurium (Te), wherein the sensor is capable of detecting one or more volatile organic compounds (VOCs) derived from one or more pathogens.

2. The sensor of claim 1, wherein each sensor is of a porous structure selected from mesoporous, macroporous, microporous, nanoporous, and hierarchical porous, wherein a pore size of the sensor material ranges from 0.4 to 2 nm, 2 to 50 nm, 50 nm to 200 nm, 200 nm to 500 nm, 500 nm to 1 μm, and 1 to 50 μm.

3. The sensor of claim 1, wherein each sensor material comprises a nanomaterial selected from nanosphere, nanorods, nanowire, nanoporous, nanomesh, nanodot, nanostar, nanosheet, nanoplate, nanoflake, nanotube, nano-hollow sphere, nanocube, nanocage, nanopolyhedron, nanobelt, nanocylinder, nanohelix, nanoprism, nanoribbon, nanoring, nanotetrapod, nanodumbbell, nanocrescent, nanosculpture, nanobrush, nanofiber, nanocrystal, nanowhisker, nanoscroll, and nanocapsule.

4. The sensor of claim 1, wherein each sensor material is selected from Pt nanospheres, Au nanodots, Ag nanowires, Ag—Au nanocubes, Os nanorods, Fe nanoparticles, Pd nanowire, Pt—Ru nanocube, Pt—Ru nanocrystal, and Ru nanocage.

5. The sensor of claim 1, wherein each sensor material comprises one or more oxides selected from CoOx, SnOx, ZnOx, NiOx, VOx, FeOx, CuOx, CrOx, InOx, TbOx, MnOx, WOx, TiOx, AlOx, GaOx, AgOx, AuOx, PdOx, RhOx, RuOx, MoOx, NbOx, ZrOx, YOx, LaOx, PtOx, SiOx, CeOx, TeOx, AgAlOx, AgAuOx, AgCoOx, AgCrOx, AgCuOx, AgFeOx, AgGaOx, AgInOx, AgMnOx, AgMoOx, AgNiOx, AgPdOx, AgPtOx, AgRhOx, AgRuOx, AgTiOx, AgVOx, AgWOx, AgZnOx, AgZrOx, AlCoOx, AlCrOx, AlCuOx, AlFeOx, AlGaOx, AlInOx, AlMnOx, AlMoOx, AlNiOx, AlPdOx, AlPtOx, AlRhOx, AlRuOx, AlTiOx, AlVOx, AlWOx, AlZnOx, AlZrOx, AuCoOx, AuCrOx, AuCuOx, AuFeOx, AuGaOx, AuInOx, AuMnOx, AuMoOx, AuNiOx, AuPdOx, AuPtOx, AuRhOx, AuRuOx, AuTiOx, AuVOx, AuWOx, AuZnOx, AuZrOx, CoCrOx, CoCuOx, CoFeOx, CoGaOx, CoInOx, CoMnOx, CoMoOx, CoNiOx, CoPdOx, CoPtOx, CoRhOx, CoRuOx, CoTiOx, CoVOx, CoWOx, CoZnOx, CoZrOx, CrCuOx, CrFeOx, CrGaOx, CrInOx, CrMnOx, CrMoOx, CrNiOx, CrPdOx, CrPtOx, CrRhOx, CrRuOx, CrTiOx, CrVOx, CrWOx, CrZnOx, CrZrOx, CuFeOx, CuGaOx, CuInOx, CuMnOx, CuMoOx, CuNiOx, CuPdOx, CuPtOx, CuRhOx, CuRuOx, CuTiOx, CuVOx, CuWOx, CuZnOx, CuZrOx, FeGaOx, FeInOx, FeMnOx, FeMoOx, FeNiOx, FePdOx, FePtOx, FeRhOx, FeRuOx, FeTiOx, FeVOx, FeWOx, FeZnOx, FeZrOx, GaInOx, GaMnOx, GaMoOx, GaNiOx, GaPdOx, GaPtOx, GaRhOx, GaRuOx, GaTiOx, GaVOx, GaWOx, GaZnOx, GaZrOx, InMnOx, InMoOx, InNiOx, InPdOx, InPtOx, InRhOx, InRuOx, AlFeTiOx, AlGaFeOx, AlGaMnOx, AlGaTiOx, AlInZnOx, AlNiZnOx, AlTiVxOx, AlVZnOx, AlZnCuOx, AlZnFeOx, AlZnMnOx, AlZnNiOx, AlZnTiOx, CoCrFeOx, CoCrMnOx, CoCrNiOx, CoCrTiOx, CoFeTiOx, CoMnNiOx, CoMnTiOx, CoNiTiOx, CoTiVxOx, CoTiZrOx, CoVZnOx, CrFeTiOx, CrMnTiOx, CrNiTiOx, FeMnTiOx, FeTiVxOx, FeTiZrOx, FeVZnOx, GaInZnOx, GaMnTiOx, GaTiZrOx, GaVZnOx, InMnZnOx, InNiZnOx, InTiZrOx, InVZnOx, MnNiZnOx, MnTiVxOx, MnTiZrOx, MnVZnOx, NiTiVxOx, NiTiZrOx, NiVZnOx, TiVZrOx, TiZrZnOx, TiZrCuOx, TiZrNiOx, TiZrCoOx, TiZrMnOx, TiZrFeOx, TiZrAlOx, TiZnCuOx, TiZnFeOx, TiZnMnOx, TiZnNiOx, TiZnCoOx, TiZnCrOx, TiZnAlOx, VZrZnOx, VZrCuOx, VZrCrOx, VZrMnOx, VZrNiOx, VZrCoOx, VZrFeOx, VZrAlOx, YAlOx, YCrOx, YFeOx, YInOx, YNiOx, YTiOx, ZnCrTiOx, ZnFeTiOx, ZnMnTiOx, ZnNiTiOx, ZnTiVxOx, ZnTiZrOx, ZrCrFeOx, ZrCrMnOx, ZrCrNiOx, ZrCrTiOx, ZrFeTiOx, ZrMnNiOx, ZrMnTiOx, ZrNiTiOx, ZrTiVxOx, ZrTiZnOx, ZrVZnOx, ZrZnCuOx, ZrZnFeOx, ZrZnMnOx, ZrZnNiOx, ZrZnCoOx, ZrZnAlOx, AlCoCrTiOx, AlCoMnTiOx, AlCoNbTiOx, AlCoTaTiOx, AlCoVTiOx, AlFeCoTiOx, AlFeCrTiOx, AlFeMnTiOx, AlFeMoTiOx, AlFeNbTiOx, AlFeTaTiOx, AlFeVTiOx, AlFeZnTiOx, AlNiCoTiOx, AlNiCrTiOx, AlNiMnTiOx, AlNiMoTiOx, AlNiNbTiOx, AlNiTaTiOx, AlNiVTiOx, AlNiZnTiOx, CuCoAlTiOx, CuCoCrTiOx, CuCoMnTiOx, CuCoMoTiOx, CuCoNbTiOx, CuCoTaTiOx, CuCoVTiOx, CuCoZnTiOx, CuFeAlTiOx, CuFeCrTiOx, CuFeMnTiOx, CuFeMoTiOx, CuFeNbTiOx, CuFeTaTiOx, CuFeVTiOx, CuFeZnTiOx, CuNiAlTiOx, CuNiCoTiOx, CuNiCrTiOx, CuNiMnTiOx, CuNiMoTiOx, CuNiNbTiOx, CuNiTaTiOx, CuNiVTiOx, CuNiZnTiOx, FeCoAlTiOx, FeCoCrTiOx, FeCoMnTiOx, FeCoMoTiOx, FeCoNbTiOx, FeCoTaTiOx, FeCoVTiOx, FeCoZnTiOx, FeNiAlTiOx, FeNiCoTiOx, FeNiCrTiOx, FeNiMnTiOx, FeNiMoTiOx, FeNiNbTiOx, FeNiTaTiOx, FeNiVTiOx, FeNiZnTiOx, ZnCoAlTiOx, ZnCoCrTiOx, ZnCoMnTiOx, ZnCoMoTiOx, ZnCoNbTiOx, ZnCoTaTiOx, ZnCoVTiOx, ZnCoZnTiOx, ZnFeAlTiOx, ZnFeCrTiOx, ZnFeMnTiOx, ZnFeMoTiOx, ZnFeNbTiOx, ZnFeTaTiOx, ZnFeVTiOx, ZnFeZnTiOx, ZnNiAlTiOx, ZnNiCoTiOx, ZnNiCrTiOx, ZnNiMnTiOx, ZnNiMoTiOx, ZnNiNbTiOx, ZnNiTaTiOx, ZnNiVTiOx, and ZnNiZnTiOx.

6. The sensor of claim 1, further comprising a carbon-based material selected from carbon black, activated carbon, microporous carbon, mesoporous carbon, micro-mesoporous carbon, pyrolytic carbon, carbon nanotube, carbon nanofiber, carbon nanospheres, carbon nanosheet, carbon nanowire, carbon nanorod, graphene, graphene oxide, and reduced graphene oxide.

7. The sensor of claim 6, wherein the carbon-based material comprises a dopant selected from sulfur, nitrogen, oxygen, boron, fluorine, bromine, iodine, chlorine, phosphorus, selenium, chlorine, and tellurium.

8. The sensor of claim 6, wherein the carbon-based material is functionalized with one or more organic agents selected from amines, fatty acids, alcohols, thiols, aldehydes, phenols, esters, epoxy, polymers, silane coupling agents, carboxylic acids, nitriles, sulfonic acids, imides, isocyanates, ketones, amides, nucleic acids, amino acids, and mixtures thereof.

9. The sensor of claim 1, wherein at least one of the one or more metal in the sensor material is a single-atom metal residing in a carbon framework.

10. The sensor of claim 1, wherein at least one of the one or more sensor materials is a composite containing an elemental metal and a metal oxide.

11. The sensor of claim 9, wherein at least one of the one or more sensor materials comprises one or more conjugated polymers selected from polypyrrole (PPy), polyaniline (PANI), polythiophene (PTh), poly(3,4-ethylenedioxythiophene) (PEDOT), poly(3-hexylthiophene) (P3HT), polyacetylene, polyphenylene vinylene (PPV), polyfluorene, polysulfone, polyindole, polyparaphenylene (PPP), and polycarbazole.

12. The sensor of claim 1, wherein the one or more VOCs are selected form acetaldehyde, acetone, acetic acid, acetophenone, ammonia, benzaldehyde, butanal, butane, butanol, carbon dioxide (CO₂), carbon disulfide, capric acid, caproic acid, caprylic acid, chlorine, decane, dimethyl ether, dimethyl pyrazine, dimethyl sulfide, dimethyl trisulfide, ethane, ethanol, ethyl acetate, formaldehyde, hexane, heptane, hydrogen cyanide, hydrogen peroxide, hydrogen sulfide, indole, isobutyric acid, isoprene, isovaleric acid, lauric acid, linoleic acid, methane, methanol, methyl cyclohexane, methyl propanoate, methyl butanoate, methyl mercaptan, methyl thiocyanate, methyl thiolacetate, myristic acid, nitric oxide, nitrogen dioxide, nonane, octane, ozone, palmitic acid, pentafluoropropionamide, pentane, propane, propionic acid, propanol, stearic acid, sulfur dioxide, 2-aminoacetophenone, 2-butanol, 2-butanone, 2-ethylhexanol, 2-heptanone, 2-methoxy-5-methylthiophene, 2-methylbutanal, 2-methylbutanol, 2-methylbutyl 2-methylbutyrate, 2-methylbutyl isobutyrate, 2-methyl-3-(2-propenyl)-pyrazine, 2-nonanone, 2-pentene, 2-pentanol, 2-tridecenone, 3-(ethylthio)-propanal, 3-hydroxy-2-butanone, 3-methyl-1-butanol, 3-methylbutanal, 3-methylbutanoic acid, 3-methyl-1H-pyrrole, 6-methyl-5-hepten-2-one, 6-tridecane, 1,1,2,2-tetrachloroethane, 1-hydroxy-2-propanone, 1-Octanol, 1-undecane, dodecane, isobutyric acid, isopropyl acetate, and valeric acid.

13. The sensor of claim 1, wherein the one or more pathogens are selected from Acinetobacter anitratus, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Ehrlichia chaffeensis, Enterobacter cloacae, Enterococcus avium, Enterococcus durans, Enterococcus faecalis, Enterococcus faecium, Enterococcus galllinamm, Enterococcus maloratus, Escherichia coli, Francisella tularensis, Fusobacterium nucleatum, Enterobacter species, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumonia, Lactobacillus acidophilus, Lactobacillus bulgaricus, Lactobacillus casei, Lactobacillus delbrueckii, Lactococcus lactis, Legionella pneumophila, Listeria monocytogenes, Methanobacterium extroquens, Microbacterium multiforme, Micrococcus luteus, Moraxella catarrhalis, Morganella morganii, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Pasteurella multocida, Pasteurella tularensis, Porphyromonas gingivalis, Prevotella melaninogenica, Proteus vulgaris, Proteus mirabilis, Proteus penneri, Providencia stuartii, Pseudomonas aeruginosa, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Yersinia enterocolitica, Yersinia pestis and Yersinia pseudotuberculosis, and/or known to comprise one or more antibiotic-resistant strains descending from a known species, and/or known to comprise one or more extended spectrum beta-lactamase-producing strains descending from a known species, in particular the one or more extended spectrum beta-lactamase-producing strain is selected from the group consisting of: extended spectrum beta-lactamase-producing Escherichia coli, and extended spectrum beta-lactamase-producing Klebsiella pneumoniae.

14. A sensor device, comprising one or more sensors of claim 1 connected to a plurality of electrodes supported on a substrate to form an electrical conduit.

15. The sensor device of claim 14, further comprising a device for measuring changes in resistance, conductance, alternating current (AC), frequency, capacitance, impedance, inductance, mobility, electrical potential, optical property or voltage threshold.

16. The sensor device of claim 14, comprising an array of sensors having 2 to 48 sensors, wherein each sensor is identical or different.
